(19) United States
(12) Patent Application Publication
(10) Pub. No.: US 2024/0295485 A1
(43) Pub. Date: Sep. 5, 2024

(54) UV SPECTROPHOTOMETRIC DETECTION MODULE OF POLYMER PARTICLES AND PHYTOPLANKTON FOR AN AUTONOMOUS WATER ANALYSIS STATION AND DETECTION PROCESS

(71) Applicant: LUXEMBOURG INSTITUTE OF SCIENCE AND TECHNOLOGY (LIST), Esch/ Alzette (LU)

(72) Inventors: Jérôme POLESEL, Esch/Alzette (LU); Olivier BOUTON, Esch/Alzette (LU)

(21) Appl. No.: 18/569,831

(22) PCT Filed: Jun. 14, 2022
(86) PCT No.: PCT/EP2022/066117

§ 371 Date: Dec. 13, 2023

(30) Foreign Application Priority Data
Jun. 14, 2021 (LU) LU500276

(51) Int. Cl. G01N 15/1429; G01N 15/00; G01N 15/1434; G01N 21/05; G01N 21/63; G01N 21/64; G01N 33/18

(52) U.S. Cl. CPC G01N 15/1429; G01N 15/00; G01N 15/1434; G01N 33/1826; G01N 33/1886; G01N 2015/0053; G01N 21/05; G01N 2021/052; G01N 2021/635; G01N 21/643; G01N 21/6486; G01N 2201/0218; G01N 2201/062

(57) ABSTRACT

A detection module of an autonomous detection system for in situ monitoring floating polymer particles and phytoplankton in sea water. The detection module is combined with a floating body, and a communication module with an antenna, and is configured for detecting polymer particles and phytoplankton such as algae. The detection module comprises: a detection area for a water sample, notably a salty water flow with polymer particles and/or phytoplankton; ultraviolet light emitting means configured for emitting ultraviolet light in the detection area, light sensing means configured for sensing light from the detection area in order to detect polymer particles and/or phytoplankton such as cyanobacteria; an energy generating module configured for powering said detection module. The UV emitting means comprise a low power consumption UV LED. A detection process, and a use of an UV light source for removing a biolayer in the detection area.

### TECHNICAL FIELD

The invention lies in the field of autonomous water monitoring, for instance to water quality assessment. In particular the invention relates to polymer and phytoplankton, such as microalgae detection, at or immediately below a water surface.

### BACKGROUND OF THE INVENTION

Nowadays the increasing presence of microplastics in the sea represents a major problem of contamination for ecological niches that is breaking the balance of many ecosystems of oceanic fauna and flora. The problem has gained attention at a global level, requiring urgent countermeasures in order to reduce the negative impact on the environment. In 2008, the EU Marine Strategy Framework Directive (MSFD, 2008/56/EC) has included “microplastics” in the list of the most relevant parameters to measure when monitoring environment.

Environmental monitoring in marine environments is critical and full of challenges. Coasts and the open sea are monitored by several satellite systems that provide insightful research data but that also reveals the need for more detailed information. Current research techniques and experimental studies for detecting microplastics in open seawaters mainly rely on Earth Observation, EO, data. Such techniques are limited by the spatial-resolution of the EO data with respect to the size of microplastics, also designated as polymer particles.

There is a vast volume of literature about microplastics analysis methods as reported in the book of Blair Crawford et al. [Elsevier, 2016, ISBN 978-0-12-809406-8, Blair Crawford, 2017]. Therein, the definition of microplastics in term of size, shapes and materials is defined. The chapter 10 “Microplastic identification techniques” of this book gives also an exhaustive overview of the different techniques for analysing these microplastics. Most of them are based on in situ sampling followed by ex situ in-labs analysis by optical or electron microscopy, Pyrolysis-gas chromatography-mass spectrometry, Nuclear magnetic resonance, NMR, spectroscopy, Fourier-transform infrared, FTIR, spectroscopy, Raman spectroscopy and Fluorescence spectroscopy.

But few of these techniques represent a good perspective in terms of integration for in field-testing or portable system with low power consumption, robustness, fast acquisition and high sensitivity. Using known systems, the monitoring cost is prohibitive on a large scale. Several solutions fail to offer a satisfying signal-to-noise ratio level, and fail to allow fast acquisition of signals of few microplastic particles taken in real time on the water flow.

### Technical Problem to be Solved

It is an objective of the invention to present a device, which overcomes at least some of the disadvantages of the prior art. In particular, it is an objective of the invention to improve a detection system.

### SUMMARY OF THE INVENTION

In accordance with a first aspect of the invention, a detection module configured for detecting polymer particles and/or phytoplankton in water is provided. The detection module comprises a translucent tubular flow-through element housing a detection area configured for collecting water at a water surface, such as a water flow with polymer particles and/or phytoplankton. The detection module further comprises ultraviolet light emitting means configured for emitting ultraviolet light in the detection area and light sensing means configured for sensing light of the detection area in order to detect polymer particles and/or phytoplankton in said detection area.

Preferably the detection module may be configured for detecting polymer particles and/or phytoplankton at or immediately below a water surface.

Preferably, the ultraviolet light emitting means and the light sensing means are configured for implementing a system for detecting floating particles and/or phytoplankton by fluorescence and/or photoluminescence.

The detection module may preferably comprise an energy generating module configured for powering the ultraviolet light emitting means and the light sensing means.

Both ends of the translucent tubular flow-through element may preferably comprise funnel-shaped openings for enlarging the water surface area that enters the tubular element.

According to another aspect of the invention it is provided a detection module configured for detecting polymer particles and/or phytoplankton in water, the detection module comprising: a detection area for water, such as a water flow with polymer particles and/or phytoplankton; ultraviolet source configured for emitting ultraviolet light in the detection area; light sensing means configured for sensing light of the detection area in order to detect polymer particles and/or phytoplankton in said detection area; an energy generating module configured for powering the ultraviolet light emitting means and the light sensing means.

Preferably, the detection module may comprise an energy generating module configured for powering the ultraviolet light emitting means the light sensing means.

Preferably, the light sensing means may be configured for sensing wavelengths ranging from 410 nm to 940 nm.

Preferably, the ultraviolet light emitting means may comprise an ultraviolet A-band light source, ultraviolet B-band light source and/or ultraviolet C-band light source, said light source(s) preferably being light emitting diode(s).

Preferably, the detection module may be configured such that the light sensing means sense wavelengths in a first wavelength range which is separate from a second wavelength range of the light emitted by the ultraviolet light emitting means.

Preferably, the light sensing means may comprise at least: 6, or 12 or 18 sensing cells configured for sensing different wavelengths of the detection area, said different wavelengths optionally comprising a constant wavelength gap.

Preferably, the detection module may comprise a machine learning device which is coupled to the light sensing means and which is configured for identifying a polymer particle and/or phytoplankton depending on a sensing signal of the light sensing means.

Preferably, the machine learning device may be configured for identifying at least: a polypropylene particle, a polyethylene particle, a polyvinylidene fluoride particle, a polycarbonate particle, polyurethane particle, a polymethyl methacrylate particle, a polystyrene particle, a polyethylene terephthalate particle, a polyamide 6 particle, a polyamide 66 particle, cyanobacteria, blue green algae, diatoms, dinoflagellates, a polymer particle with a biofilm of phytoplankton; depending on at least one wavelength sensed by the light sensing means.

Preferably, the detection module may comprise an optical filter arranged between the ultraviolet light emitting means and the light sensing means.

Preferably, the detection module may comprise light reflecting means, the detection area being arranged between the light reflecting means and the light sensing means.

Preferably, the detection module may comprise a translucent tubular element housing the detection area, the ultraviolet light emitting means being configured for emitting ultraviolet light in said transparent tubular element, wherein the transparent tubular element may preferably comprise quartz or fused silica.

Preferably, the detection area may comprise a watertight surface or enclosure optionally with a titanium oxide coating, the ultraviolet light emitting means being configured for emitting ultraviolet light through said titanium oxide coating.

Preferably, the watertight surface may comprise a roughness; such as a Ra roughness, of at most: 5 μm, or 1 μm.

Preferably, the energy generating module may comprise at least one of the following: a solar panel device, a wind turbine, a water turbine, a pivoting arm coupled to an electric generator, and any combination thereof.

Preferably, the watertight surface may comprise a roughness; such as a Ra roughness, of at most: 5 μm, or 1 μm.

Preferably, the phytoplankton may be microalgae, optionally with a size of at most: 70 μm, or 15 μm, or 5 μm, or 2 μm.

Preferably, the ultraviolet light emitting means and the light sensing means may comprise, and/or may operate on, separate and/or distinct wavelength ranges.

Preferably, the light sensing means may comprise an electronic shutter.

Preferably, the light sensing means may be configured for sensing different wavelengths than the ultraviolet light emitted by the ultraviolet light emitting means.

Preferably, the detection system may be a floating detection system, configured for floating at a water surface, or a submersible detection system.

Preferably, the light emitting means may comprise an emission wavelength of at most: 340 nm, or 315 nm, or 280 nm.

Preferably, the light emitting means may comprise a minimum emission wavelength of at least 255 nm.

Preferably, the light sensing means may be configured for sensing a wavelength of at least: 400 nm, or 410 nm.

It is another aspect of the invention to provide a detection system comprising a detection module wherein the detection module is in accordance with an aspect of the invention, and the detection system comprises a floating body with a floating line preferably substantially level with the detection area, said body optionally being a floating body.

Preferably, the detection area may comprise a water passage through the detection system, the water passage comprising an inlet and an outlet, each of the inlet and the outlet comprising a filtering mesh. Preferably the water passage may allow water to enter the detection area, Preferably, said detection system may comprise a communication module with an antenna, optionally configured for communicating with a similar detection system, and/or a communication transceiver and/or wireless gateway.

Preferably, the body may be a floating body comprising a watertight enclosure in which an energy generating module and/or the ultraviolet light emitting means and/or the light sensing means are arranged.

Preferably, the detection system may comprise a waterline, the detection area being arranged substantially at, or under the waterline, preferably at distance from said waterline.

It is another aspect of the invention to provide a pollution detection system for water, such as sea water or river water or lake water or water of a water treatment plant, the detection system comprising:
an energy generating module configured for converting energy of the environment of the detecting system into electric power;
a pollution detection module adapted to be powered by the energy producing module, the pollution detection module comprising:
   an analysis zone adapted for receiving polluted water,
   ultraviolet light emission means, such as an ultraviolet ray source, configured for illuminating the polluted water in the analysis zone, preferably for illuminating the polluting particles and/or polluting phytoplankton,
   light sensing means configured for sensing light from the analysis zone,
   an analysing unit configured for receiving a signal from the light sensing means and for computing a pollution presence score depending on said signal.

The analysis zone may be a detection area.

It is another aspect of the invention to provide a particle detection system, such as polymer particles and/or phytoplankton in water, the detection system comprising:
a body,
a detection module comprising:
   a detection area for water, such as a water flow with particles;
   ultraviolet light emitting means configured for emitting ultraviolet light in the detection area,
   light sensing means;
classifying means configured for classifying the particle(s) in at least one of the following categories, and/or for identifying at least: polypropylene material, polyethylene, polyvinylidene fluoride material, polycarbonate material, polymethyl methacrylate material, polystyrene material, polyethylene terephthalate material, polyamide 6 material, polyamide 66 material, phytoplankton; depending on at least one wavelength sensed by the light sensing means in the detection area.
an energy generating module configured for powering the detection module, notably the ultraviolet light emitting means, the light sensing means, and the classifying means.

The energy generating module is not an essential aspect of the invention.

It is another aspect of the invention to provide a detection module configured for detecting polymer particles and/or phytoplankton, the detection module comprising:
detection area for water, such as a water flow with polymer particles and/or phytoplankton and/or polymer particles coated with phytoplankton biofilms;
ultraviolet light emitting means configured for emitting ultraviolet light in the detection area;
light sensing means configured for analysing light from the water, respectively the water flow, over a predefined wavelength range.

It is another aspect of the invention to provide a detection process of polymer particles and/or phytoplankton in water with a detection module comprising a detection area, the detection process comprising the steps of: installing the detection module in water of an environment, optionally water comprising floating polymer particles and/or phytoplankton; obtaining analysed water at the detection area; converting energy from said environment into electric energy; emitting ultraviolet light in the analysed water; sensing light of the analysed water in order to detect polymer particles and/or phytoplankton in said analysed water, the detection module optionally being in accordance with the invention and the phytoplankton comprises microalgae, and/or the detection module is part of a detection system in accordance with the invention.

Preferably, the step of sensing light may comprise sensing several wavelengths of light from the analysed water.

Preferably, the detection process may further comprise a step of classifying the a detected polymer particle in one of the following material categories: polypropylene material, polyethylene, polyvinylidene fluoride material, polycarbonate material, polyurethane material, polymethyl methacrylate material, polystyrene material, polyethylene terephthalate material, polyamide 6 material, polyamide 66 material, a polymer particle with a biofilm of phytoplankton; depending light sensed at the step of sensing.

Preferably, the detection process may comprise a step of classifying phytoplankton in one of the following categories: cyanobacteria, blue green algae, diatoms, dinoflagellates; depending light sensed at the step of sensing.

Preferably, the process may comprise a step of removing a bio layer, such as a bio layer of phytoplankton, with said ultraviolet light emitting means, at the step of emitting the ultraviolet emitting means are powered with a first power; and at the step of removing, the ultraviolet emitting means are powered with a second power which is greater than the first power.

Preferably, the process may further comprise a step of filtering light from the analysed water in order to block light comprising a wavelength up to 400 nm.

Preferably, the step of emitting may last at most: 1 s, or 0.7 s.

Preferably, the process may further comprise a step of counting polymer particles in the analysed water detection area.

Preferably, the process may further comprise a step of refining the classification of a detected polymer particle in a material sub category.

Preferably, the or a step of removing the bio layer and/or algae may last at least one hour.

Preferably, the polymer particles may be floating polymer particles.

Preferably, the detection process may further comprise a step of computing a signature of the detected polymer particle(s).

Preferably, the process may comprise a step of filtering the emitted ultraviolet light at the step of emitting, and analysing the resulting light after the step of filtering.

Preferably, the process may further comprise a step of sending a detection signal.

Preferably, the body may define the detection area.

Preferably, at the step of emitting, the ultraviolet emitting means may generate a first luminous intensity; and at the step of removing, the ultraviolet emitting means may generate a second luminous intensity which is greater than the first luminous intensity.

It is another aspect of the invention to provide a water monitoring process with a particle and/or phytoplankton detection system, said detection system comprising a monitoring area such as a detection area and energy generating means, the detection process comprising the steps of:
installing the detection system in water, optionally comprising polymer particles and/or phytoplankton,
receiving water in the monitoring area, preferably receiving a water sample with polymer particles and/or phytoplankton in the monitoring area; or flowing water through the detection system;
producing electric energy with energy production means of the detection system, the produced electric power optionally being above a power threshold,
emitting ultraviolet light through water in the detection area,
detection light from water in the detection area
analysing light from water in the detection area,
generating a particle signal if the analysed and/or detected light meets a predefined criterion,
the detection system optionally being in accordance with the invention.

Preferably, the process may comprises a step of ascertaining a difference between a reference light and the detected light.

It is another aspect of the invention to provide a detection process of particles, such as polymer particles and/or phytoplankton, in water with a detection module, preferably a detection system, the detection process comprising the steps of:
installing the detection module, preferably the detection system, optionally in an environment with water; said detection module, preferably the detection system comprising energy generating means and a detection area for a water target;
producing electric energy with the energy production means by means of the environment energy,
emitting ultraviolet light in the water target by means of the electric energy produced at the step of producing electric energy,
analysing light of the water target; the detection system optionally being in accordance with the invention, and/or the detection module optionally being in accordance with the invention.

It is another aspect of the invention to provide a use of an ultraviolet light emitting means of a detection module for removing a bio layer in a polymer particle and/or phytoplankton detection module, in water, said detection module comprising light sensing means and an energy generating module which are functionally coupled to said ultraviolet light emitting means, the detection module optionally being in accordance with the invention.

Preferably, the ultraviolet light emitting means may comprise an ultraviolet light emitting diode which optionally is an ultraviolet C-band light source.

It is another aspect of the invention to provide a use of ultraviolet light emitting means of a polymer particle and/or phytoplankton, detection module, preferably an autonomous floating detection system, for removing a bio layer in said detection module, said detection module further comprising light sensing means functionally coupled to said ultraviolet light emitting means, and an energy producing module powering said ultraviolet light source and the light sensing means.

Preferably, the ultraviolet light emitting means may be used for removing a bio layer in a transparent element of said detection module.

It is another aspect of the invention to provide a use of an ultraviolet light source for detecting polymer particles and/or phytoplankton, optionally floating polymer particles and/or phytoplankton, in a detection module, said detection module notably being in accordance with the invention.

It is another aspect of the invention to provide a computer program comprising computer readable code means, which when run on a computer, cause the computer to run the detection process in accordance with the invention.

It is another aspect of the invention to provide a computer program product including a computer readable medium on which the computer program according the invention is stored.

It is another aspect of the invention to provide a computer configured for carrying out the monitoring process according to the invention.

The different aspects of the invention may be combined to each other. In addition, the preferable features of each aspect of the invention may be combined with the other aspects of the invention, unless the contrary is explicitly mentioned.

### Technical Advantages of the Invention

A majority of plastics have a density typically lower than that of water. This is for example the case of polyethylene—the most abundant plastic found in the aquatic environment. With good water-repellent characteristics, polyethylene microplastics is typically found floating on surface waters. Additionally, foamed, or expanded, varieties of polystyrene are often found floating on the surface of aquatic environments. The density of a microplastic as a key factor that affects its spatial distribution in an aquatic environment. In a study involving the collection of microplastics by neuston nets from the North Atlantic, microplastics with a greater density than that of seawater were found floating on the surface waters. Similarly, in another study of plastic litter on surface waters, 99% of the microplastic recovered by neuston nets in the western North Atlantic Ocean had an average density less than that of seawater, with the density of the microplastics ranging from 0.808 to 1.238 g/cm3. Incidentally, the density of seawater is considered to be 1.025 g/cm3. These results may be surprising since intuitively, one would not typically expect to find material floating on the surface with a density greater than that of seawater. Thus, it is possible that microplastics with a density significantly greater than that of seawater can be found on surface waters, albeit in small quantities, and not exclusively in the bottom sediment. There are two main reasons as to why this can occur: first, the occurrence of such high density microplastics in surface waters can result from powerful upward and downward movements of water, as result of temperature differences at different depths (vertical mixing), and second, microplastics that are more dense than seawater may contain pockets or bubbles of air within them, thereby increasing their buoyancy and allowing them to float on the surface.

Providing a solution to analyse floating biological objects such as algae, phytoplankton is highly relevant because the greater part of these living micro-objects with a photosynthesis-based metabolism (e.g. cyanobacteria, blue algae) seek the water surface to be both in the liquid medium of survival and to be exposed to the greatest density of light. Thus, they have developed buoyancy by their density and/or by their surface energy The invention provides such a detection module that is configured for detecting floating polymer particles and phytoplankton at or immediately beneath a water surface. The specific arrangement of the detection area allows to collect water at its surface, where the relevant particles are floating. The invention also provides a detection process of polymer particles and phytoplankton. The invention offers a use of an ultraviolet light source notably coupled to a visible near-infrared (NIR) sensor for photoluminescence and fluorescence analysis. A general benefit of the invention is the ability to provide a low-power consumption detection module system, which allows it to be used in an autonomous system, wherein power is scarce. The invention offers an in-situ solution with detection means which provide accurate results based on a low power consumption per measurement cycle. Data acquisition, data treatment and wireless transmission of the treated data only require a low energy level.

This aspect is of prime relevance in the context of an autonomous detection system, notably equipped with its own energy production module which supply power for the other energy consuming modules. As a result, the monitoring module remains autonomous during month or years.

The invention is adapted for both polymer particles and phytoplankton. Thus, it offers accurate observations of different kinds of pollutions which are noxious for health. This benefit may be obtained by photoluminescence and fluorescence analyses. Thereby, the invention extends the technical capacities of the detection module, respectively the detection system. By using a specifically designed water collection tube, water at the surface, which carries floating particles or organisms, is efficiently analysed.

### BRIEF DESCRIPTION OF THE DRAWINGS

Several embodiments of the present invention are illustrated by way of figures, which do not limit the scope of the invention, wherein

FIG. 1 provides a schematic illustration of a detection system in accordance with a preferred embodiment of the invention;

FIG. 2 provides a schematic illustration of a detection module in accordance with a preferred embodiment of the invention;

FIG. 3 provides a schematic illustration of a detection module of a detection system in accordance with a preferred embodiment of the invention;

FIG. 4 provides a schematic illustration of a detection process in accordance with a preferred embodiment of the invention;

FIG. 5 provides a schematic illustration of wavelengths measured by a detection module in accordance with a preferred embodiment of the invention when a particle of a thin foil transparent bag is in the detection area;

FIG. 6 provides a schematic illustration of wavelengths measured by a detection module in accordance with a preferred embodiment of the invention when a particle of transparent blue plastic PMMA is in the detection area;

FIG. 7 provides a schematic illustration of wavelengths measured by a detection module in accordance with a preferred embodiment of the invention when a particle of PU dark blue foam is in the detection area;

FIG. 8 provides a schematic illustration of wavelengths measured by a detection module in accordance with a preferred embodiment of the invention when a particle of PU white foam is in the detection area;

FIG. 9 provides a schematic illustration of wavelengths measured by a detection module in accordance with a preferred embodiment of the invention when a particle of PU orange foam is in the detection area;

FIG. 10 provides a schematic illustration of wavelengths measured by a detection module in accordance with a preferred embodiment of the invention when a particle of a thick foil transparent bag is in the detection area;

FIG. 11 provides a schematic illustration of wavelengths measured by a detection module in accordance with a preferred embodiment of the invention when a particle of polystyrene bead is in the detection area;

FIG. 12 provides a schematic illustration of wavelengths measured by a detection module in accordance with a preferred embodiment of the invention when a particle of PU beige foam is in the detection area;

FIG. 13 provides a schematic illustration of a graph illustrating the light intensity depending on the wavelength measured by a detection module in accordance with a preferred embodiment of the invention;

FIG. 14 provides a schematic illustration of a graph illustrating the light intensity depending on the wavelength measured by a detection module in accordance with a preferred embodiment of the invention;

FIG. 15 provides a schematic illustration of a graph illustrating the light intensity depending on the wavelength measured by a detection module in accordance with a preferred embodiment of the invention.

### DETAILED DESCRIPTION OF THE INVENTION

This section describes the invention in further detail based on preferred embodiments and on the figures. Similar reference numbers will be used to describe similar or the same concepts throughout different embodiments of the invention.

It should be noted that features described for a specific embodiment described herein may be combined with the features of other embodiments unless the contrary is explicitly mentioned. Features commonly known in the art will not be explicitly mentioned for the sake of focusing on the features that are specific to the invention.

It should be understood that the ability of a particle to float is impacted by several properties: the water density, the particle density, the particle porosity, and the surface energy defining the hydrophobicity of the particle material. The density difference between freshwater and seawater is small to note alone an impact on floatability. The main impact on the floatability concerns the salt concentration inducing changes of the surface energy of water: 60-65 dynes/cm2 for seawater, 72 dynes/cm2 for freshwater. Pollutants at the water surface may induce higher surface energy and wettability of the polymer particles. The pollutants notably encompass oils or soap. So, considering the water and the seawater for both cases the plastic particles will have a close behaviour of floatability only dependent of external parameters (oil, soap, biofilms coating) inducing drastic change on floatability.

It should be understood that the floating polymer particles are not limited to particles touching the water surface. In choppy sea, floating particle may go deep in water, under the water surface. Thus, the detection system is sensitive to floating particles at distance from the water surface.

Throughout the invention, the term phytoplankton may designate microscopic organisms living, and/or developing, in watery environments, both salty water and fresh water.

The term “microalgae” may designate single cell organisms, for instance drifting in water. It could be understood that microalgae comprise chlorophyll.

In the current description, the ultraviolet light corresponds to a form of electromagnetic radiation between visible light and X-rays.

Except when the contrary is explicitly provided, a particle refers to a bare particle; namely a particle free of outer layer such as a bio coating or a phytoplankton coating.

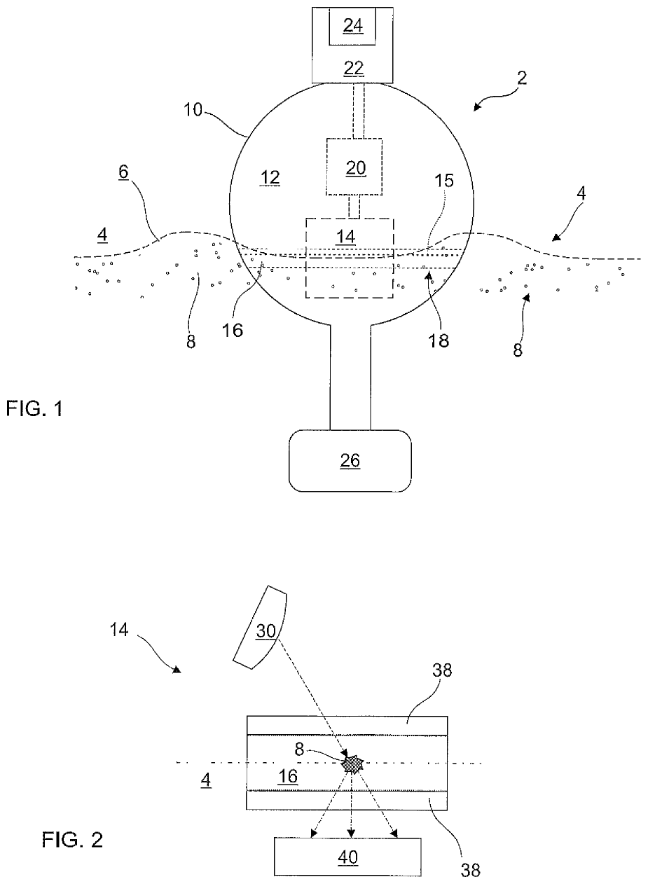

FIG. 1 shows a detection system 2 in accordance with a preferred embodiment of the invention. In the current illustration, the detection system 2 is floating in water 4, notably in open water. The detection system 2 may be arranged in sea water, in a river, in a lake, in a pond, or a water treatment plant. Other water areas are considered. The water 4 is essentially in a liquid state. It is aqueous. Due to wind, to flow, the detection system 2 is exposed to waves 4 at the water surface 6.

The water is charged with particles 8, such as floating particles 8; and/or phytoplankton. Phytoplankton such as microalgae may be mixed in water. The particles may be polymer particles 8 floating at the surface 6. The polymer particles 8 may be designated as plastic particles 8.

The system 2 exhibits a buoyant body 10. The body 10 is a floating body. The buoyant body 10 may define an enclosure 12. The enclosure 12 is water tight, and ensures the capacity of the system 2 to stay at the water surface 6. Due to waves, the body oscillate about an equilibrium orientation.

The enclosure 12 houses, receives, different modules of the detection system 2. The enclosure 12 protects a detection module 14. The body 10 may form a casing protecting the detection module 14. The detection module 14 is structurally and functionally configured for identifying and detecting particles, notably polymer particles 8 and/or phytoplankton such as micro algae by a method of fluorescence and/or photoluminescence.

The polymer particles 8 are suspended in water 4. Due to their density/densities, the polymer particles 8 are immersed in water 4, at the water surface 6. The polymer particles 8 may comprise a size ranging from 1 mm to 5 mm. The size of the polymer particles 8 may correspond to the greatest dimension of said polymer particles 8. It may be a maximum width, or a maximum diameter. The body 10 defines a waterline 15, also designated as floating line. The waterline 15 may be considered as a fictions line. At rest, the waterline 15 may be drawn by a horizontal plane cutting the body 10. The detection module 14 may be vertically level with the waterline 15, so that water at the surface may flow through the detection module, which comprises a substantially horizontally arranged flow-through tubular element to that effect.

The phytoplankton may be mixed in water 4. The phytoplankton is not represented as such due to its size. The phytoplankton may be microalgae, with a size measured at the micrometer scale. The size may range from: 0.5 μm to 60 μm, or from 0.8 μm to 1.2 μm. The phytoplankton may comprise cyanobacteria and bacterial spores.

The detection system 2 comprises a detection area 16 associated with the detection module 14. The detection module 14 may be a passage 18, such as a water passage 18. The passage 18 is provided by a flow-through tubular element having translucent walls and preferably having a circular or rectangular cross-section. The passage 18 may cross the detection system 2, notably the body 10. It may form a passage through the enclosure 12. The passage 18 may comprise a watertight inner surface. The passage 18 may be formed by a tight wall, extending across the body 10. The water passage 18 may form a channel in the buoyant body 10 adapted to collect, to obtain, an analysed water volume or water target. The water passage 18 is generally immersed and may emerge due to waves and tilting motions of the body 10. Preferably, the water passage is substantially level with the waterline 15.

The detection area 16 may generally be at the water level, or generally under the surface 6. As a consequence, water 4 enters in the detection area 16, and flows through said passage 18. Then, the particles of interest and the phytoplankton are driven in the detection area 16 for analysis purposes. As an option, the detection area 16 is under the waterline 15. The body 10 may comprise a vertical separation between the detection area 16 and the waterline 15. Thus, more water enters in the detection area 16, and may avoid that air enters therein. This provision increases the detection accuracy.

The detection system 2 further comprises a power module, such as an energy generating module 20. Alternatively, a charged battery may be used to provide power. The energy generating module 20 is configured for electrically feeding, at least, the detection module 14. The energy generating module 20 may convert energy from the environment and generates usable electric power. The energy generating module 20 may be an embedded power harvester with smart power management means for regulated output voltage. Hence the detection system 2 is energy autonomous. The energy produced is self-sufficient; it covers the system's needs over its whole service life. If required, the system may enter in a stand-by mode when energy is missing. It will reactivate when the energy level of the energy accumulator reaches a threshold. By way of illustration, the smart power management means may be configured for regulating AC-to-DC energy stored in a tank such as a supercapacitor, a battery. Then power is delivered by DC-to-DC converter to provide a regulated voltage level; typically: 3.3 V or 5V; to the detection module 14.

The energy generating module 20 may use water energy, sun energy, and/or wind energy as a primary source. Water energy embraces wave energy, flow energy such as from tides. The energy generating module 20 may comprise or be replaced a battery pack accumulating electric power, and/or a super capacitor. An external electric power supply may replace the energy generating module.

The energy generating module 20 may include a single pendulum, or a double pendulum. The energy generating module 20 may include a wind turbine (not represented). The system 2 may form the floating base of the wind turbine. As an option, the system 2 combines at least two energy production solutions. As yet another option, the system is electrically connected to a solar panel (not represented). The top of the system 2 may form the floating base of the solar panel installation which is exposed to the sun, thereby combining two energy production solutions. A 10 cm*10 cm solar panel is sufficient. The wave energy source and the sun energy source offer the same electric power level: about 100 mW. These energy sources provide energy level which are in the same order of magnitude, with the advantage of the electromagnetic generator of the pendulum to work over the whole day; notably during the night since the movements of waves is continuous. Also, the electromagnetic generator of the double pendulum protected inside the body 10 is robust against corrosion and bio layer formation. The latter could be limiting for an external solar panel solution depending on optical transmission from the solar light as degraded by the presence of a masking bio layer.

As an option, the detection system 2 further comprises a communication module 22. The communication module 22 may generally form communication means, for instance for emitting data related to the water 4 analysed in the water passage 18, the status of the water surface 6 movements (calm sea or choppy sea) and/or the geolocation of the system 2. The water status may be provided through an embedded accelerometer module and/or a gyroscope module and/or a magnetometer module (not represented). The communication module 22 may comprise an antenna 24. The antenna 24 may be on top of the body 10 or enclosed in the body 10. The antenna 24 may form the summit of the system 2. The communication means may comprise a wireless communication module and a microcontroller associated with the antenna 24.

The communication module 22 may be configured for wireless communication. The communication module 22 may transmitting signal to remote communication infrastructures (not represented), for instance airborne and/or ground based infrastructures (not represented). The communication module 22 may be configured for communication with similar or identical detection systems (not represented). The similar or identical detection systems may define a communication network.

As a further option, the detection system 2 may comprise a ballast 26 adapted for maintaining a predefined orientation. The ballast 26 may comprise a block at the bottom of the detection system 2. The ballast 26 may generally be means for orienting said system 2 it in a predefined and privileged orientation when the system 2 is floating. It also maintains the detection area 16 in a specific orientation with respect to the water surface 6. As an alternative, the detection system 2 comprises a moor (not represented). Said moor means may be fixation means. The fixation means may comprise a fixation hole. These solutions are convenient in light of ocean's tides.

The system in accordance with the invention may be considered as a floating station 2 for water analysis. The station 2 may be an autonomous floating analysis station. The station is adapted for different kinds of analysis, such as pollutant objects detection in water, status of the sea waves. The station 2 may further comprise a location module (not represented) for providing a location signal of the buoyant body in the sea. The location signal may be computed by means of at least one of the following: a magnetometer module, a compass module, a GPS signal and combination thereof. Water pollution may be detected and located. Specific particles may be observed. Some biological species may be observed. An associated map may be established.

FIG. 2 shows an illustration of a detection module 14 in accordance with a preferred embodiment of the invention. The detection module 14 comprises a translucent tubular flow-through element 38 housing a detection area 16 configured for collecting water 4 at a water surface, such as a water flow with polymer particles 8 and/or phytoplankton. When floating in water and/or arranged in a detection system such as the one shown in FIG. 1 (but not limited thereto), the flow-through tube 38 is substantially horizontal and level with the surface of the water 4. This allows a skim of particles floating at, or immediately beneath the water surface, to flow through the tubular element. The detection module further comprises ultraviolet light emitting means 30 configured for emitting ultraviolet light in the detection area 16 and light sensing means 40 configured for sensing light of the detection area 16 in order to detect polymer particles 8 and/or phytoplankton in said detection area 16 by a process of fluorescence and/or photoluminescence. The arrangement is preferably such that light emitted from the ultraviolet light emitting means 30 first reaches particles 8 within the deception area. This triggers the emission of light in the direction of the light sensing means 40. Following the path of emitted light, the light sensing means are preferably arranged downstream of the particles 8 and of the detection area 16. The emission and sensing means may preferably be coupled and co-ordinated through the use of an appropriately programmed microcontroller element, which is capable of activating the light emission, and reading out the light sensing means, for storing the collected data in a memory element, where it may be further processed and analysed. The length of the tubular element 38 is chosen so that it may pierce entirely through the body of a floating detection system (such as the one shown in FIG. 1) which it equips. Preferably, the length of the tubular element 38 is within the range from 10 to 20 cm. Preferably, the length is of about 15 cm, while the diameter of the tube is of at least 1 cm, and preferably of 15 to 20 mm. Preferably, the diameter is of about 16 mm.

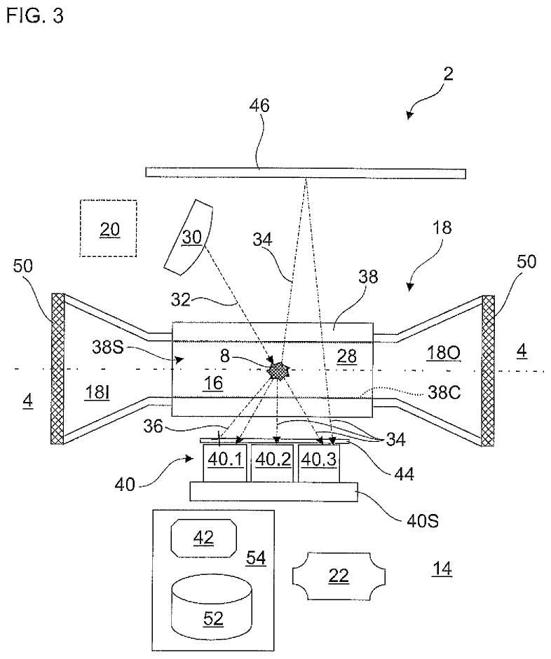

FIG. 3 shows an illustration of a detection module 14 of a detection system 2 (partially represented) in accordance with another preferred embodiment of the invention. The detection system 2 may be similar or identical to the one described in connection with FIG. 1. The detection module 14 is in fluid flow communication with the water 4 forming the environment of the detection system 2. The body and the enclosure are omitted for the sake of clarity. Water is optionally salt water, for instance with a density of 1.03 g/cm3, or more.

The detection module 14 is adapted for receiving a liquid, notably water 4. The water forms analyzed water 28, also considered as a water volume or a water flow. The analyzed water 28 may flow in the detection area 16. The detection area 16 may receive the moving analyzed water 28. The detection area 16 is crossed by a throughput or a flow of water to investigate.

The analyzed water 28 notably transports a particle 8 such as a polymer particle 8. For clarity reasons, only one particle 8 is currently represented. However, the detection area 16 may receive several polymer particles 8 simultaneously. The polymer particle 8 may be a fragment of a polymer part which has been divided up. In addition, the detection area 16, respectively the analyzed water 28, may comprise phytoplankton. The phytoplankton may be micro algae, with a micrometric scale. These phytoplankton comprise, by way of illustration, bacterial spores, with a size ranging from 0.8 μm to 1.2 μm. Microalgae modify the turbidity of water. The detection module 14 is optionally configured for measuring the water turbidity. The phytoplankton and/or the polymer particle(s) 8 may be mixed in the analyzed water 28. The polymer particles 8 may be encapsulated in a phytoplankton coating.

The system 2 is configured for detecting polymer particles 8 and/or phytoplankton in water 4. The system 2 may be an analyzing system and/or a monitoring system adapted to a watery environment. Thus, the system 2 is tight, and configured for resisting to corrosive liquids.

The detection module 14 further comprises ultraviolet (UV) light emitting means 30, also designated as an ultraviolet light source. The UV emitting means 30 are configured for emitting ultraviolet light in the detection area 16. They are adapted for emitting first UV light beams 32 toward the particle 8 and/or the phytoplankton, in, and through the detection area 16. The first UV light beams 32 meet the particle 8. The first UV light beams 32 are also considered as emitted beams 32, primary beams 32, or source beams 32. The first UV light beams 32 have a first wavelength range ranging from 100 nm to 400 nm. The first UV light beams 32 may comprise UV-A, and/or UV-B, and/or UV-C light beams, with wavelengths ranging from: 100 nm to 280 nm; 280 nm to 315 nm; 315 nm to 400 nm; respectively. The ultraviolet light emitting means 30 comprise an ultraviolet A-band light source, an ultraviolet B-band light source and/or an ultraviolet C-band light source, said light source(s) preferably being UV light emitting diode(s) (LEDs). The UV LEDs are convenient since they require a low power consumption. As another solution, a UV laser could be used. When the first light beams 32 strike the polymer particle 8, second light beams 34 are emitted. The second light beams 34 are also considered as secondary beams 34, or response beams 34. The second light beams 34 can have different wavelengths than the first light beams 32. They may have a second wavelength range which differ from the first wavelength range. The second wavelength range may be shifted with respect to the first wavelength range. It may be broader. The second light beams 34 have wavelengths varying from, at least: 410 nm to 940 nm. The detection system 2 is configured such that the light sensing means 40 sense wavelengths in a so called/the second wavelength range which is separate, and optionally distinct, from a so called/the first wavelength range of the light emitted by the ultraviolet light emitting means 30. Similarly, when the first light beams 32 meet phytoplankton, the second light beams 34 are emitted and sensed by the sensing means 40. The light sensing means 40 may be wavelength measuring means. The system 2 is configured for protecting the sensing means 40 from the sunlight. Indeed, the sunlight generates a background signal.

As an option, third light beams 36 are emitted as well by the polymer particle 8 and/or phytoplankton. The third light beams 36 have different wavelengths than the first and second UV light beams 34.

The detection area 16 may be formed by a tubular element or tube 38. The tube 38 may a transparent tube 38. The tube 38 is also designated as transparent tubular element 38. It may generally be a transparent element housing/defining the detection area 16. As a channel, the tube 38 houses and encircles the detection area 16. It physically guides the analyzed water. The ultraviolet light emitting means 30 are configured for emitting ultraviolet light in and through said transparent tubular element 38, across the water target.

As an option, the transparent tubular element 38 comprises quartz or fused silica. These materials exposed to UV light prevent a biolayer buildup. Hence, the transparent tubular element 38 keeps its optical transparency over time. Thus, the polymer particles and phytoplankton observations are safeguarded over time.

As a further option, the tube 38 comprises a coating 38C. The coating is an inner coating 38C. The detection area 16, notably the tube 38, comprises a tight surface 38S. The tight surface 38 is also designated as inner surface 38. It is water tight. The surface 38 comprises the coating 38C. The coating 38 may be a titanium oxide coating (TiO2). The coating 38 C accelerates photolysis of a disturbing organic biofilm. The nature of the coating limits and avoids, biofouling in the detection area 16. The biofouling is considered as a natural growth, notably an algae growth. The ultraviolet light emitting means 30 are configured for emitting ultraviolet beams 32 through said titanium oxide coating 38C. It may be a transparent coating 38C. The secondary beams 34 also cross the coating 38C.

Still as an option, the transparent tubular element 38 comprises an inner roughness, Ra, of at most: 10 μm, or 3 μm, or 1 μm. The Ra roughness may be understood as the arithmetical mean deviation of the assessed profile. The reduced roughness avoids and reduces the biolayer buildup on the inner surface 38S. The adhesion, the grip, on the inner surface 38S is reduced. This contributes to the fact that the detection area 16 remains clean, and that a maintenance free character 2 is conferred on the system.

The detection module 14 comprises light sensing means 40. The light sensing means 40 are configured for sensing light in the detection area in order to detect polymer particles 8 and/or phytoplankton in said detection area 16. The energy generating module 20 is adapted for powering the detection module 14. The energy generating module 20 provides electric power to the light sensing means 40 and the light emitting means 30 as well.

The light sensing means 40 are configured for sensing wavelengths ranging from 410 nm to 940 nm. On the one hand, the lower limit of 410 nm (blue/violet colour) is defined also in order to avoid overlap with the UV emission light; even UV LEDs have a broad range emission spectrum with small emission in the violet colour. The system 2 may be configured such that it comprises a wavelength separation between the first wavelength range and the second wavelength range. On the other hand, the invention considers shifting the higher limit of 940 nm (Near Infra Red=NIR). It may be shifted at least: to 1500 nm, or to 2000 nm. An upper limit may be selected due to light absorption by water with high loss of the signal. A range from 410 nm to 940 nm represents a good compromise in the context of water analysis and optical transmittance, and of the chemical compounds of interest.

The sensing means 40 may comprise at least one sensing unit, preferably at least three sensing units (40.1; 40.2; 40.3). Each sensing unit (40.1; 40.2; 40.3) may comprise several sensing cells (not represented), for instance at least 6 sensing cells (not represented). The light sensing means 40 comprise at least: 6, or 12 or 18 sensing cells configured for sensing different wavelengths of the detection area 16. The number of sensing cells offers a compromise between accuracy, sensitivity, fast data treatment and energy consumption; the latter being an important factor in the context of an energy autonomous system 2.

The more sensors to explore the UV-visible-NIR spectrum the better the definition of the peaks for the microplastics recognition. But more sensors imply a higher power consumption and more signal treatments; which is also energy consuming. The configuration with 3×6=18 cells represents a good compromise between resolution, power consumption, data treatment, acquisition speed. The resolution is tailored to both polymer particles and phytoplankton. As a non-limiting example, the AS7265× Smart Spectral Sensor from the company AMS AG may be used.

Each sensing cell is associated with one predefined wavelength. The light sensing means 40 may comprise a constant gap between the predefined wavelengths. The light sensing means 40 may comprise a support 40S receiving the sensing units (40.1; 40.2; 40.3), in front of the detection area 16.

As an option, the detection module 14 comprises a machine learning device 42 which is coupled to the light sensing means and which is configured for identifying a polymer particle 8 and/or phytoplankton depending on a sensing signal emitted by the light sensing means 40. The sensing signal may form a sensing signature which is compared to a reference signature. The machine learning device 42 may be a microcontroller containing the neuronal network matrix to treat the data flow from the light sensor 40.

The machine learning device 42 may implement a machine learning algorithm trained in order to detect, identify: polymer particles, phytoplankton, polymer particles covered by a phytoplankton layer. The machine learning algorithm, or machine learning model, is trained with learning data, and tested with test data. The machine learning algorithm is selected when a loss, or error, meets a predefined criterion.

The machine learning device may comprise a neural network. The neural network may be a feedforward network. As an option, the neural network may comprise an input layer, an output layer, and hidden layers connecting the input layer to the output layer. The hidden layers may be fully connected, or sparsely connected. The input layer may comprise several input neurons. As an option, each sensing cell of the light sensing cell is associated with one input neuron. Thus, the input layer may comprise from 6 to 18 input neurons. In the context of the invention implementing a machine learning algorithm, from 6 to 18 sensing cells is a relevant compromise between accuracy, sensitivity, fast data treatment and energy consumption. Energy consumption is due to measures and signal processing. The output layer may comprise several output neurons, notably at least: one per polymer particle kind and one per microalgae kind. Each output neuron may provide a probability, such as detection probability, or an identification probability. The hidden layers may comprise rows of hidden neurons. As an option, the neural network is a convolutional neural network, implementing a convolution kernel.

The machine learning device 42 may be configured for classifying the polymer particle 8 in at least one of the following particle categories: polypropylene particle, polyethylene particle, polyvinylidene fluoride particle, polycarbonate particle, polymethyl methacrylate particle, polystyrene particle, polyethylene terephthalate particle, polyamide 6 particle, polyamide 66 particle, Polyvinylidene fluoride particle. Other polymer categories are considered. The machine learning device 42 may be further configured for identifying phytoplankton such as cyanobacteria in the detection area 16. Identification may essentially rely on secondary wavelengths sensed by the light sensing means 40.

The detection module 14 may comprise an optical filter 44 between the ultraviolet light emitting means 30 and the light sensing means 40. The optical filter 44 may be between the detection area 16 and the light sensing means 40. The optical filter 44 allows passage of the second visible-NIR light beams 34. The optical filter 44 may block, as a mask, the third UV light beams 36. The optical filter 44 is configured for blocking lighting under a predefined wavelength, for instance of 410 nm. It protects the sensing units (40.1; 40.2; 40.3) from beams outside or under a wavelength range of interest. The optical filter 44 may absorb or reflect the third UV light beams 36. The latter may come from the environment of the system 2.

The detection module 14 may comprise light reflecting means 46, such as a mirror. The detection area 16 extends between the light reflecting means 46 and the light sensing means 40. The ultraviolet light emitting means 30 are arranged between the light reflecting means 46 and the detection area 16. Thus, the light sensing means 40 receive more secondary beams 34 from the particle 8 and the detected phytoplankton. The detection is more accurate. The signal-to-noise ratio of the measurements is improved; allowing shorter acquisition periods. The system 2 is adapted to a fast motion of the particle 8.

The detection area 16 comprises a water passage 18 through the detection system 2. The water passage 18 may be formed by the tube 38. It comprises an inlet 181 and an outlet 180. The ends of the tube may preferably be funnel-shaped, as depicted. This increases the aperture of the tube openings, and allows for particles floating on a larger surface area of water to be collected by the flow-through tube 38. The water passage 18 is reversible, as water may flow in both directions therethrough. Each of the inlet 181 and the outlet 180 comprise a filtering mesh 50. The filtering meshes 50 avoids clogging. It physically prevents big bodies to enter in the system 2. Such big bodies could shut and cut the passage 18, such that no water circulation would occur. No detection would be performed. The filtering meshes 50 may comprise holes of at most 6 mm. The filtering meshes 50 execute a selection of polymer particle 8 undergoing analyses.

The monitoring device may comprise a data storage 52, such as computer readable medium 52. The data storage 52 may store a computer program adapted for executing a detection process in accordance with the invention. The data storage 52 may be embedded in a microcontroller unit (MCU) 54. The microcontroller unit 54 preferably comprises a data processor, such as a central processing unit, CPU, of a computing device. The data processor may preferably be programmed by appropriately formulated software code to implement the method steps.

The computer readable medium 52 may comprise a Random-Access Memory, RAM module, or a persistent storage device such as EEPROM (electrically erasable programmable read-only memory), a Hard Disk Drive, HDD, or a Solid-State Drive, SSD.

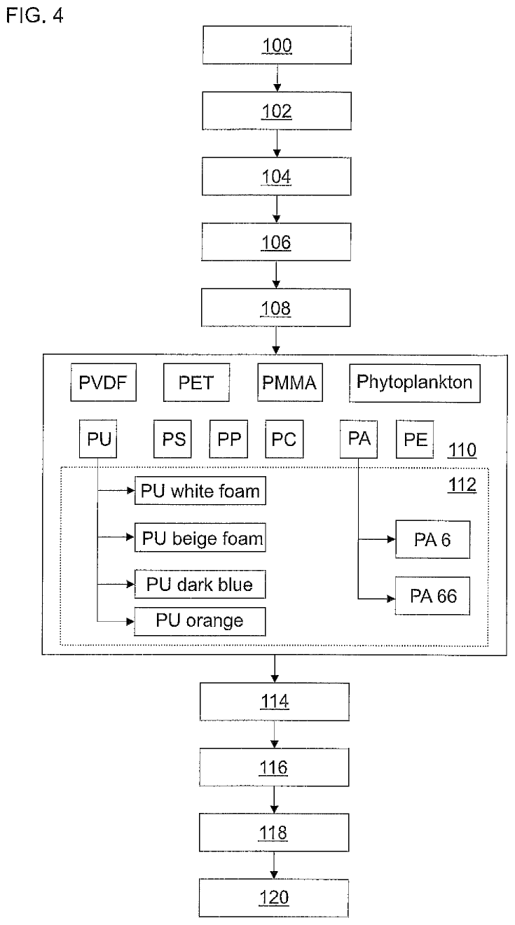

FIG. 4 provides a schematic illustration of a diagram of a detection process. The detection process enables detection of polymer particles and phytoplankton, such as cyanobacteria generally designated as blue-green algae. The phytoplankton may comprise diatoms. The phytoplankton may comprise a unicellular or a multicellular organism. It may develop or occur in fresh or salt water. It may be observed in underground water. The detection process may be executed by means of a detection module; optionally as defined in FIGS. 1 and/or 2. It may be executed by means of a detection module, optionally a detection system, notably as described in FIGS. 1 and/or 2.

The detection process comprises the steps of; notably executed in the following sequence:
installing 100 the detection module; optionally the detection system; in water of an environment, such as ocean water comprising floating polymer particles and/or phytoplankton;
obtaining 102 analysed water, or obtained water, or receiving water, at the detection area; converting 104 energy from the module environment, respectively the system environment, into usable electric energy;
emitting 106 ultraviolet light in the analysed water, also designated as water target;
sensing 108 light of the water sample in order to detect polymer particles and/or phytoplankton such as microalgae in said water target;
classifying 110 a polymer particle in at least one of the following categories: polypropylene (PP) particle, polyethylene (PE) particles, polyvinylidene fluoride (PVDF) particle, polycarbonate (PC) particle, polymethyl methacrylate (PMMA) particle, polystyrene (PS) particle, polyethylene terephthalate (PET) particle, polyamide particle, polyurethane (PU) particle; depending light sensed at the step of sensing 108. As an alternative, micro algae, is detected and then classified.

At the step of installing 100, the system may be arranged in sea or ocean. It may be arranged in a river, a lake, a pond. It may be in a plant, producing or treating water. It may drift freely. As an option, it is fixed to a support. Since the detection system may be drifting, as a free buoy, the steps may be executed at different locations.

The step of emitting 106 lasts at most: 1 s, or 0.7 s. Hence, power consumption is limited. At the step of emitting 106 the ultraviolet light, UV-A light, UV-B light, and/or UV-C light are emitted through the analysed water, toward the hypothetical polymer particle therein. These UV lights can be emitted simultaneously.

The step of sensing 108 may comprise a sub step of detecting (not represented) polymer particles and/or phytoplankton.

The step of sensing light 108 comprises sensing several wavelengths of light from the water sample. The wavelengths are sensed simultaneously.

The step of classifying 110 may comprise the detection of phytoplankton, such as microalgae. The process may comprise the step of detecting the polymer particles and/or phytoplankton between the steps of sensing 108 and the step of classifying 110. The step of detecting may be an intermediate step.

The process may further comprise a step of refining 112 the classification of a polymer particle and/or phytoplankton. The step of refining 112 may replace the step of sensing 108. The step of refining 112 may comprise the sub classification of a PU particle in one of the following subcategories: polyurethane dark blue foam, polyurethane white foam, polyurethane orange foam, polyurethane beige foam. Different colours of dyes contained in the polymer particles may be distinguished. The step of refining 112 may be a step of colour detection.

The step of of refining 112 may comprise the sub classification of a detected polyamide particle PA in one of the following subcategories: polyamide 6 (PA-6) or polyamide 66 (PA-66).

At the step of classifying 110, a specific material may be detected, for instance a polymer particle of a bag.

At the step of refining 112, the particle may be further classified depending on its thickness. The particle may be classified in a first thickness category, or in a second thickness category which is thicker than the former.

The process comprises a step of removing 114 a bio layer, such as an algae layer, in the detection area 16 with the ultraviolet light emitting means. For this purpose, at the step of emitting 106, the ultraviolet emitting means are powered with a first power, and at the step of removing 114 the ultraviolet emitting means are powered with a second power which is greater than the first power. The first power may generate a first lighting intensity, and the second power may generate a second lighting intensity. The light intensities may correspond to illumination flux, or UV beam concentrations. The step of removing 114 may be longer than the step of emitting 106, for instance at least: 10 or 100 times longer. It may last one hour.

The process may comprise a step of filtering 116 light from the water volume in order to block light comprising a wavelength up to 400 nm.

The detection process may further comprise a step of counting 118 polymer particles in the detection area.

The number of particles may be stored in a data storage device.

The process may comprise a step of sending 120 a signal. The signal may comprise data related to detected or counted polymer particles, the location of the system, the number and the kind of polymer particle observed. This signal can also contain a warning flag in case of detection of toxic species for flora and fauna.

The current process may correspond to a monitoring process, such as a water quality monitoring process. Features defined in relation with the detection system may specifically apply to the detection module.

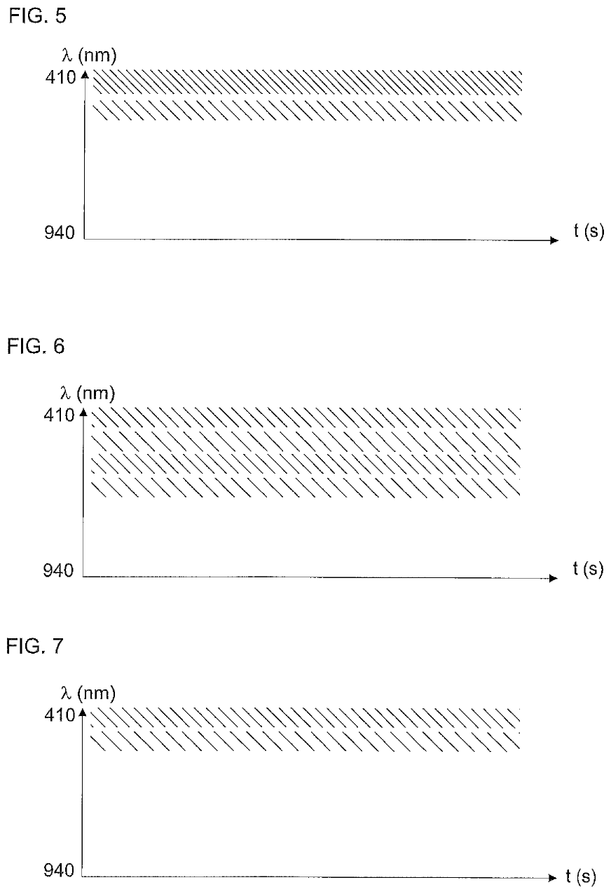

FIG. 5 provides a schematic illustration of wavelengths (A) test measures by the detection module, notably of a detection system. Wavelength measures are executed over predefined time duration, for instance with a fixed time interval between subsequent measurement points. The detection module may be as described in the previous figures. The wavelengths (A) are expressed in nanometres (nm). In the current illustration and following ones, a detection system prototype is installed in a waves' pool, and polymer particle samples are introduced in said prototype. The detection system prototype may be similar or identical to the system(s) described in the previous figures. The artificial waves move water through the detection system, and contribute to drive the polymer particles during their detection. Accordingly, the test is more realistic.

The light intensities are represented with different hatchings, for wavelengths (A) from 410 nm to 940 nm. The illustration provides a denser hatching for greater intensities. The lower intensities are represented with loose hatchings. It will be understood that in practice, the light intensities exhibit smooth gradations.

During measures, waves may be generated in the waves' pool. The polymer particles are transported by water through the detection area, notably the water passage. The polymer particles comprise a size between: 0.5 mm to 10 mm, or 1 mm and 5 mm; values included. The polymer particle motions may result from surface flow, for instance driven by waves.

Still for the sake of testing the system, polymer particles are fixed to an optically neutral or a string crossing the detection area. Dragging the rope through the detection module simulates the particle motion. In addition, it sets a crossing time. The sensitivity of the detection module is proven. Still for the purpose of the tests, the polymer particles are illuminated with a same UV primary light with a wavelength of 340 nm. As an option, the polymer particles are illuminated with several LED, emitting UV-A, UV-B, and UV-C light beams respectively. The detection time, including data acquisition, may last several minutes, for instance: 5 or 20 minutes with a sampling data rate of 0.7 s/sample. The same protocol is applied in the context of the following figures.

In real conditions, in nature, the polymer particles move in the detection area at different speeds. Hence, the detection period or presence period may vary. It may be understood from the current figure that several particles are detected over the analysis period. As an alternative a same polymer particle may be detected several times due to flow inversions, or when the flow stops.

As apparent from the current graph, the polymer particles of thin foil transparent bags, when illuminated with an UV light source, generate lights with different wavelengths, spreading on the second wavelength range. The current wavelengths are obtained when polymer particles of a thin foil transparent bag are in the detection area. The detection system, by means of the light sensing means, detects a peak at 435 nm. The emitted light essentially ranges from 410 nm to 460 nm. An auxiliary peak is sensed at 510 nm.

FIG. 6 provides a schematic illustration of wavelength time series measured by a detection module, notably of a detection system. The detection module may be as described in the previous figures. The detection system may be similar or identical to at least one or those described in the previous figures. The current wavelengths (A) are obtained when polymer particles of transparent blue PMMA (Poly(methyl methacrylate)) are in the detection area.

The detection module detects a main peak at 435 nm. By contrast with the previous graph associated with particles of thin foil transparent bag, the main peak is lower: less powerful. The detection module also detects a secondary peak at 560 nm and a third peak at 645 nm.

Thus, secondary light emitted by the polymer particles of transparent blue PMMA differs from light emitted by the polymer particles of thin foil transparent bag, albeit an exposure to a same primary UV lighting. This difference is used for detection purpose, and also for classifying.

FIG. 7 provides a schematic illustration of temporal wavelengths (A) measured by a detection module, notably of a detection system. The detection module may be as described in the previous figures. The detection system may be similar or identical to the one(s) as described in the previous figure(s). The current wavelengths are obtained for polymer particles of PU (Polyurethane) dark blue foam.

The detection module measures, by means of its light sensing cells, different wavelengths. The detection module identifies a main intensity peak at 460 nm. Other low intensities are observed close-by. These data deviate from the previous by a generally lower intensity level. Intensity variation over time (s) is detected.

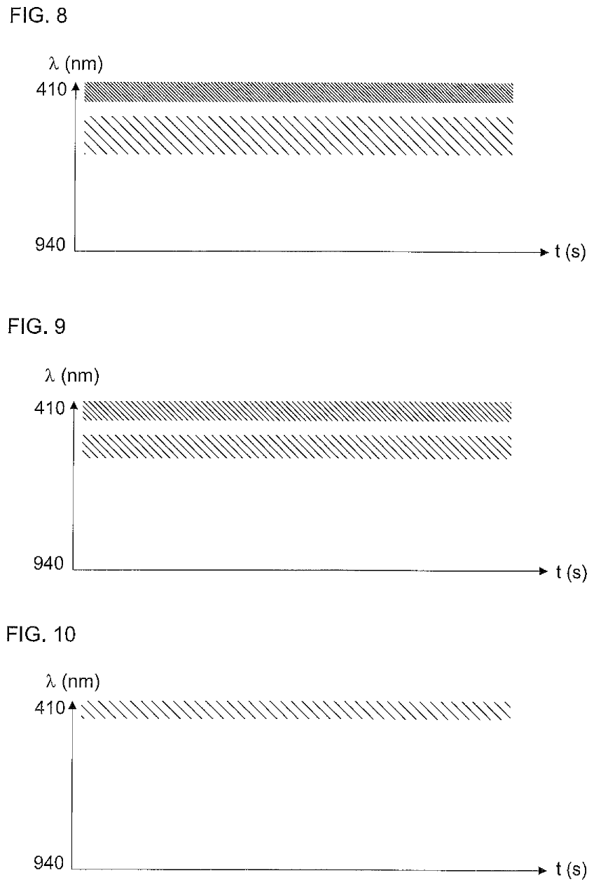

FIG. 8 provides a schematic illustration of wavelengths (A) measured during a period of detection by a detection module, notably of a detection system. The detection module may be as described in the previous figures. The detection system may be similar or identical to the one(s) as described in the previous figure(s). The current wavelengths are obtained for polymer particles of PU white foam.

The detection module measures a high peak at 435 nm. This main peak includes a summit at 435 nm, and ranges from 410 nm to 460 nm. This peak is higher than the peak detected in connection with thin foil transparent bag. A secondary peak is observed between 510 nm and 535 nm, and slightly decrease until 585 nm.

Wavelengths are detected at 705 nm at a low intensity level.

FIG. 9 provides a schematic illustration of wavelengths (A) measured by a detection module, notably of a detection system. The detection module may be as described in the previous figures. The detection system may be similar or identical to the one(s) as described in the previous figure(s). The current wavelengths are obtained for polymer particles of PU orange foam.

The detection module proves a main peak at 435 nm. However, its intensity lies between the particles of thin foil transparent bag and particles of PU white foam. The current main peak varies from 435 nm to 460 nm. The secondary peak extends from 510 nm to 535 nm.

The current particles are PU particles as in FIG. 6 and FIG. 7. Yet, the colors differ. The invention is able to detect this difference, and to refine classification depending on the color. The invention allows a classification is sub-categories. The invention enables a classification depending on the colors of the particles.

FIG. 10 provides a schematic illustration of wavelengths (A) measured by a detection module, notably of a detection system. The detection module may be as described in the previous figures. The detection system may be similar or identical to the one(s) as described in the previous figure(s). The current wavelengths are obtained when polymer particles of a thick foil transparent bag are in the detection area.

The polymer particles of a thick foil transparent bag are thicker than the polymer particles of the thin foil transparent bag presented in relation with FIG. 4. They may be of the same colour. They may be at least: two, or four, times thicker. The polymer particles of the thin foil transparent bag, also designated as first particles, may comprise a thickness ranging from: 20 μm to 40 μm, or 30 μm to 40 μm. They may have a thickness of 35 μm, optionally of at most 35 μm. The polymer particles of a thick foil transparent bag, also designated as second particles, may comprise a thickness ranging from: 50 μm to 90 μm, or 60 μm to 80 μm, or 50 μm to 70 μm. The second particles may comprise a thickness of at least 70 μm.

The detection module tracks light intensities for different wavelengths (A) over time. A main peak at 435 nm is highlighted. Its intensity is higher than with PMMA, and lower than with respect to the thin foil transparent bag. Thus, the invention operates a distinction depending on the particle thickness. This distinction grounds again a sub-classification. The invention allows for refining the step of sorting.

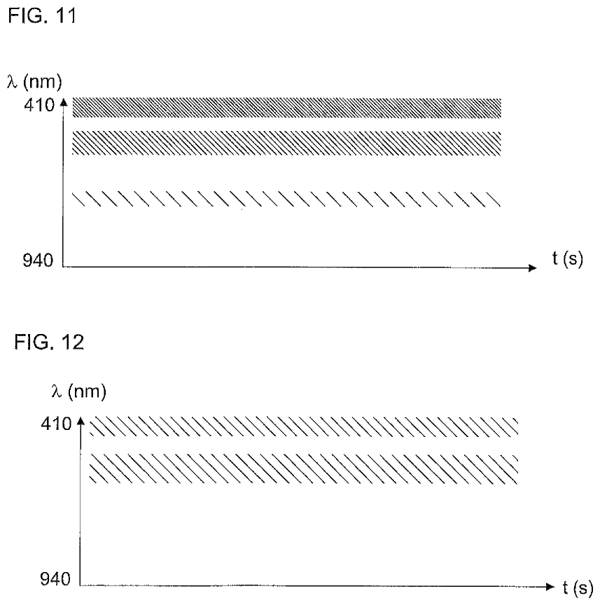

FIG. 11 provides a schematic illustration of wavelengths (A) measured by a detection module, notably of a detection system. The detection module may be as described in the previous figures. The detection system may be similar or identical to the one(s) as described in the previous figure(s). The current wavelengths (A) are obtained for polymer particles of polystyrene beads.

The particles may have different shapes. They may form flakes, or beads. They may be essentially flat or spherical.

The detection module obtains a main peak at 435 nm. This main peak includes a summit at 435 nm, and ranges from 435 nm to 460 nm. By comparison with PU white foam, the summit is shifted.

A secondary peak is observed between 510 nm and 535 nm. With respect to the secondary peak obtained of PU white foam, it is higher and narrower.

Small light signal is also sensed between 680 nm and 705 nm.

FIG. 12 provides a schematic illustration of wavelengths (A) measured by a detection module, notably of a detection system. The detection module may be as described in the previous figures. The detection system may be similar or identical to the one(s) as described in the previous figure(s). The current wavelengths are obtained for polymer particles of PU beige foam.

The detection signal from the detection signal includes wavelength data. The wavelength data includes a main peak ranging from 410 nm to 460 nm, and a summit at 435 nm. The secondary peak ranges from 510 nm to 535 nm. The intensity footprint has a similar profile than the PU orange foam. Nevertheless, the intensities are lower. This illustration confirms that polymer particles of different colours generate different wavelengths which are sensed by the detection module in accordance with the invention. The previous colours do not form a limiting list. On the contrary, the invention is adapted for identifying, recognizing, classifying other colours.

Similar curves may be obtained for cotton swab. Then, a high and narrow main peak is observed at 435 nm. And adjacent peak is detected at 485 nm. A third small peak appears at 705 nm.

The previous graphs correspond to times series data. They may be obtained on a same time length. We may observe that, for the above polymer materials, the wavelengths are lower than 730 nm, preferably of at most 705 nm. The wavelengths of interest, in the context of polymer particles, range from 410 nm to 705 nm. They may be monitored by light sensing cells, for instance from 6 to 18 cells.

In FIGS. 5 to 13, the wavelengths may correspond to measures on polymer particles free of phytoplankton coating. They may be bare particles.

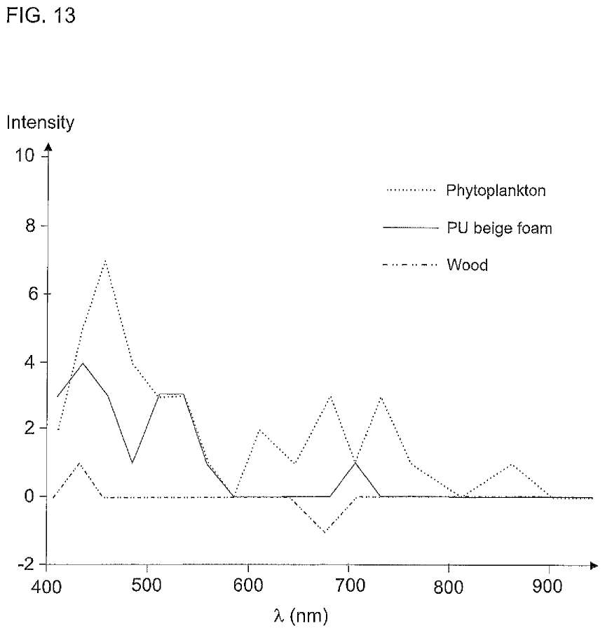

FIG. 13 provides a schematic illustration of a graph of measured light of a phytoplankton (doted lines), such as micro algae by a detection module in accordance with the invention. The current graph represents light intensities, for instance in lumen, depending on wavelengths (A) measured in nanometres (nm). The detection module may be part of a detection system corresponding to the system(s) described in at least one of the previous figures.

The graph stems from measures on a water sample in the detection area. In order to prepare a sample to analyse, limnospira cyanobacteria, also designated as blue-green algae, is diluted in tap water with a concentration of 57 ug/ml. The sample is illuminated with a UV light, for instance with a wavelength of 355 nm. Other test samples may be prepared with other phytoplankton species to investigate. The integration time of the photoelectronic sensors on board comprises a duration of 280 milliseconds by acquisition point. This acquisition time may be longer than the acquisition time for polymer particles. By way of illustration, the acquisition time for polymer particles may be of 42 milliseconds. Increasing the acquisition time in the context of phytoplankton increases the signal-to-noise ratio. Hence, detection is more accurate.

As apparent from the current graph, there is a significant light emission between 600 nm and 750 nm, which significantly differs from the primary UV light. This light emission may correspond to a Near Infra-Red (NIR) light. It may be a fluorescent light. The shape of the broken line corresponds to the presence of cyanobacteria. Indeed, the dashed line exhibits peaks and rifts which denote a proportion of limnospira cyanobacteria in the test sample.

Similar curves may be obtained for other phytoplankton, such as micro algae, which influence the water turbidity. Several cyanobacteria species can also present light emissions in the blue light due to the presence of specific proteins. The light emission in the 600 nm to 850 nm range (red to NIR) is due to the presence of Phycocyanin and Chlorophyll molecules.

It may be observed that the dashed line associated with phytoplankton is defined by 18 points. Each point may be associated with one of the 18 sensing cells. It may be derived therefrom that the lines correspond to light rays as measured by the detection module, and more precisely the light sensing means. Hence, a limited amount of data is used for featuring polymer particles and phytoplankton. However, this amount of data is sufficient for detecting, classifying, identifying the polymer particles and the phytoplankton. It may be observed that the sensing cells are each associated with wavelengths generally increasing with a constant wavelength gap.

The current figure also includes a solid line illustrating light emitted by a sample including polymer particles. The polymer particles are polyurethane (PU) particles, such as particles of beige foam polyurethane. This solid line is superposed to the measured with respect to algae. The current figure highlights the clear distinction between phytoplankton and polymer particles as enabled by the detection module of the invention.

Still for illustrative purpose, the present figure includes a broken line, with dashes and dots, illustrating measures of wood particles, for instance bare wood particles. The current graph confirms that wood does not generate a significant signal in the detection module. Hence, it does not disturb measures of the system which keeps it abilities despite perturbing bodies. The same conclusion may be drawn with respect to sand. A polymer particle may be detected beside a wood particle, or sand.

Hence, the use of an ultraviolet light source offers an interesting benefit for detecting, monitoring, analysing, water with polymer particles and/or algae. It may be observed that wavelengths for phytoplankton, such as microalgae, are essentially in two ranges: from 410 nm to 535 nm, and from 610 nm to 760 nm. The latter range marks a significant difference with polymer particles. A peak at 860 nm deepens the difference.

The current graph combines, overlays different broken lines. It confirms the differences of transmitted light by microalgae and PU beige foam, albeit a same UV light exposure.

When a polymer particle is covered by phytoplankton coating, for instance partially or totally covered by said phytoplankton coating, the detection module senses a light emission corresponding to combination of the phytoplankton and polymer reemission light wavelengths with distinct values allowing to analyse three cases: polymer particle fully covered by a phytoplankton coating, polymer particle partially covered by a phytoplankton coating, polymer particle without phytoplankton coating.

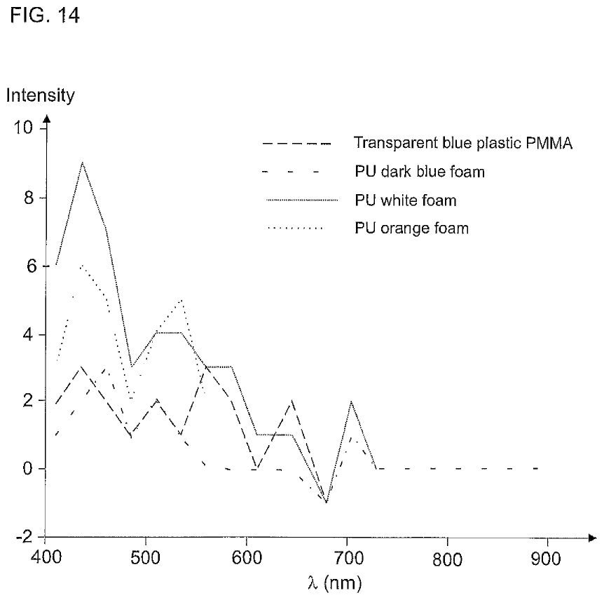

FIG. 14 provides a schematic illustration of a graph of light intensities depending on wavelengths (A). The wavelengths analysed range from 410 nm to 940 nm, which matches the wavelengths of the sensing means: the second wavelength range. The current range encompasses the visible light, and extends beyond. The detection system corresponds to the system(s) described in at least one of the previous figures.

The current graph comprises wavelengths of different PU foam materials, and the wavelengths for a transparent blue plastic, notably of PMMA. The light intensities are represented with a dedicated scale for the sake of clarity. The current scale is representative of the orders of magnitude for several wavelengths. Each wavelength may be associated with one of the sensors. The graph informs on the difference between the different PU foams as sensed by the detection module.

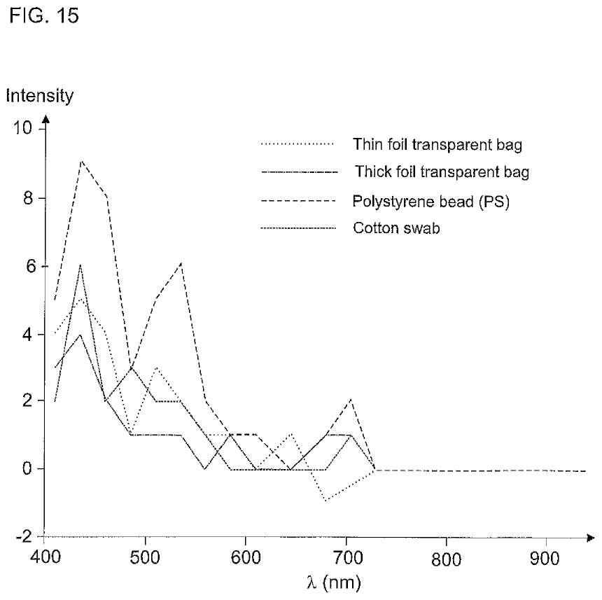

FIG. 15 provides a schematic illustration of light intensity depending on wavelengths. The broken lines represent light from polymer particles. The polymer particles may have different shapes. The wavelengths (A) are measures by a detection system. The detection system corresponds to the system(s) described in at least one of the previous figures.

The FIG. 15 focusses on the difference between particles of thin and thick foil transparent bags, preferably of the same polymer material. Even if the broken lines are close to one another along several segments, we observe deviations.

The broken lines also explicit variations between the cotton swab sample and the polystyrene bead sample. The latter may also be compared against the PU white foam of FIG. 13 in order to identify the difference between the peaks and valleys.

FIGS. 13 to 15 may have same scales. Thus, their broken lines may be overlapped for comparison.

FIGS. 5 to 12 comprise time series data. The measured intensities may vary depending on the particle speed with respect to the sensing means.

FIGS. 5 to 12 are illustrated with six hatching gradations representing at least: six intensities levels. Different light intensities may be merged in one kind of hatching.

FIGS. 13 to 15 may correspond to data of FIGS. 5 to 12. Data of FIGS. 13 to 15 may rely on averaged data obtained through wavelength intensity measures. As an alternative, it may rely on maximum light intensities. Each line corresponds to a particle or a phytoplankton signature.

The data extracted from any of FIGS. 5 to 15 may be used as training data, test data and validation data for the machine learning algorithm. These data form labelled training data. Different measures of each polymer material are used in order to create learning instances with labels. Each training instance comprise different light intensities for wavelengths from 410 nm to 940 nm. The light intensities comprise at least: 6, or 12 or 18, light intensities numerical values.

The same is carried out for phytoplankton, notably limnospira also known as a variety of blue-green algae or cyanobacteria.

Features defined in relation with phytoplankton may apply to microalgae or algae, and vice versa; except where the contrary is explicitly mentioned.

It should be understood that the detailed description of specific preferred embodiments is given by way of illustration only, since various changes and modifications within the scope of the invention will be apparent to the person skilled in the art. The scope of protection is defined by the following set of claims.

1. A detection module configured for detecting at least one of polymer particles and phytoplankton in water, the detection module comprising:
a translucent tubular flow-through element housing a detection area configured for collecting the water at a water surface, with the at least one of the polymer particles and the phytoplankton;
ultraviolet light emitting means configured for emitting ultraviolet light in the detection area; and
light sensing means configured for sensing light of the detection area in order to detect the at least one of the polymer particles and the phytoplankton in said detection area.

2. The detection module according to claim 1, further comprising an energy generating module configured for powering the ultraviolet light emitting means and the light sensing means.

3. The detection module according to claim 1, wherein both ends of the translucent tubular flow-through element comprise funnel-shaped openings for enlarging the water surface area that enters the tubular element.

4. The detection module according to claim 1, wherein the light sensing means are configured for sensing wavelengths ranging from 410 nm to 940 nm.

5. The detection module according to claim 1, wherein the ultraviolet light emitting means comprise at least one of an ultraviolet A-band light source, ultraviolet B-band light source and ultraviolet C-band light source, said ultraviolet A-band light source, said ultraviolet B-band light source and said ultraviolet C-band light source are light emitting diodes.

6. The detection module according to claim 1, wherein the detection module is configured such that the light sensing means sense wavelengths in a first wavelength range which is separate from a second wavelength range of the light emitted by the ultraviolet light emitting means.

7. The detection module according to claim 1, wherein the light sensing means comprise at least: sensing cells configured for sensing different wavelengths of the detection area, said different wavelengths optionally-comprising a constant wavelength gap.

8. The detection module according to claim 1, wherein the detection module comprises a machine learning device which is coupled to the light sensing means and which is configured for identifying the at least one polymer particle and the phytoplankton depending on a sensing signal of the light sensing means.

9. The detection module according to claim 8, wherein the machine learning device is configured for identifying at least: a polypropylene particle, a polyethylene particle, a polyvinylidene fluoride particle, a polycarbonate particle, a polyurethane particle, polymethyl methacrylate particle, a polystyrene particle, a polyethylene terephthalate particle, a polyamide 6 particle, a polyamide 66 particle, cyanobacteria, blue green algae, diatoms, dinoflagellates, a polymer particle with a biofilm of phytoplankton; depending on at least wavelengths sensed by the light sensing means.

10. The detection module according to any of claim 1, wherein the detection module comprises an optical filter between the ultraviolet light emitting means and the light sensing means.

11. The detection module according to claim 1, wherein the detection module comprises light reflecting means, the detection area being between the light reflecting means and the light sensing means.

12. The detection module according to claim 1, wherein the ultraviolet light emitting means are configured for emitting ultraviolet light in said transparent tubular element, the transparent tubular element comprising quartz or fused silica.

13. The detection module according to claim 1, wherein the detection area comprises a watertight surface with a titanium oxide coating, the ultraviolet light emitting means being configured for emitting ultraviolet light through said titanium oxide coating.

14. The detection module according to claim 13, wherein, the watertight surface defines a roughness having a Ra roughness value of 1 μm.

15. The detection module according to claim 2, wherein the energy generating module comprises at least one of the following: a solar panel device, a wind turbine, a water turbine, a pivoting arm coupled to an electric generator, and any combination thereof.

16. A detection system comprising detection module in accordance with claim 1, wherein the detection system comprises a body with a floating line substantially level with the detection area.

17. The detection system according to claim 15, wherein the openings of said translucent tubular flow-through element comprise a filtering mesh.

18. The detection system according to claim 16, wherein said detection system comprises a communication module with an antenna, configured for communicating with a similar detection system, and/or a communication transceiver and/or wireless gateway.

19. The detection system according to claim 15, wherein the body is a floating body comprising a watertight enclosure in which an energy generating module and/or the ultraviolet light emitting means and the light sensing means are arranged.

20. A detection process of polymer particles and/or phytoplankton in water with the detection module according to claim 1, the detection process comprising the steps of:
installing the detection module in the water of an environment, the water comprising floating polymer particles and/or phytoplankton;
obtaining analysed water at the detection area;
converting energy from said environment into electric energy;
emitting ultraviolet light in the analysed water; and
sensing light of the analysed water in order to detect polymer particles and/or phytoplankton in said analysed water, the phytoplankton comprising microalgae.

21. The detection process in accordance with claim 20, wherein the step of sensing light comprises sensing several wavelengths of light from the analysed water.

22. The detection process in accordance with claim 20, wherein the detection process comprises a step of classifying a polymer particle in one of the following material categories: polypropylene material, polyethylene, polyvinylidene fluoride material, polycarbonate material, polyurethane material, polymethyl methacrylate material, polystyrene material, polyethylene terephthalate material, polyamide 6 material, polyamide 66 material, a polymer particle with a biofilm of phytoplankton; depending light sensed at the step of sensing.

23. The detection process in accordance with claim 20, wherein the detection process comprises a step of classifying phytoplankton in one of the following categories: cyanobacteria, blue green algae, diatoms, dinoflagellates; depending light sensed at the step of sensing.

24. The detection process in accordance with claim 20, wherein the process comprises a step of removing a bio layer, such as a bio layer of phytoplankton, with said ultraviolet light emitting means; at the step of emitting the ultraviolet emitting means are powered with a first power; and at the step of removing, the ultraviolet emitting means are powered with a second power which is greater than the first power.

25. The detection process in accordance with claim 20, wherein the process further comprises a step of filtering light from the analysed water in order to block light comprising a wavelength of at most to 400 nm.

26. The detection process in accordance with claim 20, wherein the step of emitting lasts at most 0.7 s.

27. The detection process in accordance with claim 20, wherein the process further comprises a step of counting polymer particles in the analysed water.

28. The detection process in accordance with claim 20, wherein the process further comprises a step of refining the classification of a detected polymer particle in a material sub category.

29. Use of ultraviolet light emitting means of a detection module for removing a bio layer in said detection module which is configured for detecting polymer particles and/or phytoplankton in water; said detection module comprising light sensing means which are functionally coupled to said ultraviolet light emitting means, the detection module being in accordance with claim 1.

30. The use in accordance with claim 29, wherein the ultraviolet light emitting means comprises an ultraviolet light emitting diode which is an ultraviolet C-band light source.
